(19) United States
(12) Patent Application Publication
(10) Pub. No.: US 2012/0108445 A1
(43) Pub. Date: May 3, 2012

(54) VEGF AND VEGFR1 GENE EXPRESSION USEFUL FOR CANCER PROGNOSIS

(75) Inventor: Heinz-Josef Lenz, Los Angeles, CA (US)

(73) Assignee: UNIVERSITY OF SOUTHERN CALIFORNIA, Los Angeles, CA (US)

(21) Appl. No.: 13/265,538

(22) PCT Filed: Apr. 23, 2010
(86) PCT No.: PCT/US2010/032261

§ 371 Date: Jan. 5, 2012

Related U.S. Application Data
(60) Provisional application No. 61/172,562, filed on Apr. 24, 2009.

(51) Int. Cl. C12Q 1/68; C40B 40/06; C40B 30/00

(52) U.S. Cl. 506/7; 435/6.12; 435/6.11; 506/16

(57) ABSTRACT

The invention provides compositions and methods for determining the likelihood of tumor recurrence of adjuvant cancer patients following surgical resection.

### CROSS-REFERENCE TO RELATED APPLICATION

This application claims the benefit under 35 U.S.C. §119(e) of U.S. Provisional Ser. No. 61/172,562, filed Apr. 24, 2009, the contents of which is incorporated by reference in its entirety.

### STATEMENT AS TO FEDERALLY SPONSORED RESEARCH OR DEVELOPMENT

This invention was made with government support under the National Institutes of Health Grant P30 CA 14078. Accordingly, the U.S. Government has certain rights to the invention.

### FIELD OF THE INVENTION

This invention relates to the filed of pharmacogenomics and specifically to the application of genetic polymorphisms to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568.

Although considerable research correlating gene expression and/or polymorphisms has been reported, much work remains to be done. This invention supplements the existing body of knowledge and provides related advantages as well.

### SUMMARY OF THE INVENTION

The invention provides compositions and methods for determining the likelihood of tumor recurrence of adjuvant cancer patients following surgical resection.

Thus, in one aspect, this invention provides compositions and methods for determining the likelihood of tumor recurrence of adjuvant cancer patients following surgical resection. Thus, in one aspect, this invention provides a method for identifying an adjuvant cancer patient as more or less likely to experience tumor recurrence, comprising, or alternatively consisting essentially of, or yet further consisting of, determining an intratumoral expression level of VEGF or VEGFR1 gene in a cell or tissue sample of the corresponding cancer isolated from the patient, wherein the presence of:

(a) a high or overexpression of VEGF or VEGF gene expression level higher than a predetermined first value; or

(b) a high or overexpression of VEGFR1 gene expression level higher than a predetermined second value,

identifies the patient as more likely to experience tumor recurrence, or the presence of neither of (a) or (b) identifies the patient as less likely to experience tumor recurrence. In some embodiments, the presence of:

(c) a low or underexpression of VEGF or VEGF gene expression level lower than the predetermined first value; or

(d) a low or underexpression of VEGFR1 or VEGFR1 gene expression level lower than the predetermined second value,

identifies the patient as less likely to experience tumor recurrence.

Also provided by this invention is a method for identifying an adjuvant cancer patient as more or less likely to experience tumor recurrence, comprising, or alternatively consisting essentially of, or yet further consisting of, determining an intratumoral expression level of VEGF gene in a cell or tissue sample of the corresponding cancer isolated from the patient, wherein a high or overexpression of VEGF ro VEGF gene expression level higher than a predetermined value identifies the patient as more likely to experience tumor recurrence, or a low or underexpression of VEGF or VEGF gene expression level lower than the predetermined value identifies the patient as less likely to experience tumor recurrence.

Also provided by this invention is a method for treating a patient having a cancer by administering to the patient a therapy comprising, or alternatively consisting essentially of, or yet further consisting of an adjuvant cancer therapy, wherein the patient is selected for the therapy based on a genotype of low or underexpression of VEGF or VEGF gene expression level lower than the predetermined value identifies in a sample isolated from the patient, thereby treating the patient.

Yet further provided is a method for identifying an adjuvant cancer patient as more or less likely to experience tumor recurrence, comprising, or alternatively consisting essentially of, or yet further consisting of, determining an intratumoral expression level of VEGFR1 gene in a cell or tissue sample of the corresponding cancer isolated from the patient, wherein a high or overexpression of VEGFR1 or VEGFR1 gene expression level higher than a predetermined value identifies the patient as more likely to experience tumor recurrence, or a low or underexpression of VEGFR1 or VEGFR1 gene expression level lower than the predetermined value identifies the patient as less likely to experience tumor recurrence.

Also provided is a method for treating a patient having cancer by administering by to the patient a therapy comprising, or alternatively consisting essentially of, or yet further consisting of an adjuvant cancer therapy, wherein the patient is selected for the therapy based on a genotype of low or underexpression of VEGFR1 or VEGFR1 gene expression level lower than the predetermined value identifies the patient in a sample isolated from the patient, thereby treating the patient.

This invention also provides a kit for use in identifying an adjuvant cancer patient more likely to have tumor recurrence, comprising, or alternatively consisting essentially of, or yet further consisting of suitable primers, probes or a microarray for determining an expression level of VEGF or VEGFR1 gene, and instructions for use therein.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

The term “adjuvant” cancer patient refers to a patient to which administration of a therapy or chemotherapeutic regimen has been given after removal of a tumor by surgery, usually termed adjuvant chemotherapy. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

As used herein, the term “patient” intends an animal, a mammal or yet further a human patient. For the purpose of illustration only, a mammal includes but is not limited to a human, a simian, a murine, a bovine, an equine, a porcine or an ovine.

“5-FU based therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20(5):461-470. An example of such is an effective amount of 5-FU and Leucovorin. Other chemotherapeutics can be added, e.g., oxaliplatin or irinotecan.

Fluorouracil (5-FU) belongs to the family of therapy drugs call pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

In one aspect, the term “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

VEGF (vascular endothelial growth factor, Entrez Gene ID: 7422, UniProtKB: P15692, http://www.ncbi.nlm.nih.gov/, last accessed Apr. 17, 2009) is a member of the PDGF/VEGF growth factor family and encodes a protein that is often found as a disulfide linked homodimer. VEGF protein is a glycosylated mitogen that specifically acts on endothelial cells and has various effects, including mediating increased vascular permeability, inducing angiogenesis, vasculogenesis and endothelial cell growth, promoting cell migration, and inhibiting apoptosis. Elevated levels of this protein is linked to POEMS syndrome, also known as Crow-Fukase syndrome. Mutations in this gene have been associated with proliferative and nonproliferative diabetic retinopathy.

VEGFR1 (fms-related tyrosine kinase 1 or vascular endothelial growth factor/vascular permeability factor receptor, Entrez Gene ID: 2321, UniProtKB: P17948) is an oncogene belonging to the src gene family and is related to oncogene ROS (MIM 165020). Like other members of this family, it shows tyrosine protein kinase activity that is important for the control of cell proliferation and differentiation.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The term “genetic marker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

A “haplotype” is a set of alleles of a group of closesly linked genes which are usually inherited as a unit. The term “allelic variant of a polymorphic region of the gene of interest” refers to a region of the gene of interest having one of a plurality of nucleotide sequences found in that region of the gene in other individuals.

The term “genotype” refers to the specific allelic composition of an entire cell or a certain gene and in some aspects a specific polymorphism associated with that gene, whereas the term “phenotype’ refers to the detectable outward manifestations of a specific genotype.

“Expression” as applied to a gene, refers to the production of the mRNA transcribed from the gene, or the protein product encoded by the gene. The expression level of a gene may be determined by measuring the amount of mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

An “internal control” or “house keeping” gene refers to any constitutively or globally expressed gene. Examples of such genes include, but are not limited to, β-actin, the transferring receptor gene, GAPDH gene or equivalents thereof. In one aspect of the invention, the internal control gene is β-actin.

“Overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present invention, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

When the expression level of a gene or a genetic marker or polymorphism is used as a basis for selecting a patient for a treatment described herein, the expression level or genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more more desirable clinical outcome as compared to a patient or patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to a patient or patients having the same disease and receiving the same therapy but not possessing the characteristic. As defined herein, the patients is considered suitable for the therapy. In another such aspect, a patient possessing a characteristic may exhibit one or more more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Having the same cancer” is used when comparing one patient to another or alternatively, one patient population to another patient population. For example, the two patients or patient population will each have or be suffering from colon cancer.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

### Descriptive Embodiments

The invention further provides diagnostic, prognostic and therapeutic methods, which are based, at least in part, on determination of the expression level of a gene of interest identified herein.

For example, information obtained using the diagnostic assays described herein is useful for determining if a subject is suitable for cancer treatment of a given type or is likely to experience tumor recurrence. Based on the prognostic information, a doctor can recommend a therapeutic protocol, useful for reducing the malignant mass or tumor in the patient or treat cancer in the individual.

Determining whether a subject as more or less likely to experience tumor recurrence, alternatively, can be expressed as identifying a subject as more likely to experience tumor recurrence or identifying a subject as less likely to experience tumor recurrence.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods of this invention are useful for the diagnosis, prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer. In one aspect, the adjuvant patients are stage 2 cancer patients and had not yet received any additional therapy after surgery or surgical resection. In an alternative aspect, the adjuvant patients are stage 3 cancer patients and will receive or had received additional therapy after surgery or surgical resection. The additional therapy comprises, or alternatively consists essentially of, or yet further consists of 5-FU based adjuvant therapy.

The methods are useful in the assistance of an animal, a mammal or yet further a human patient. For the purpose of illustration only, a mammal includes but is not limited to a simian, a murine, a bovine, an equine, a porcine or an ovine.

### Diagnostic Methods

This invention provides a method for identifying an adjuvant cancer patient as more or less likely to experience tumor recurrence, comprising, or alternatively consisting essentially of, or yet further consisting of, determining an intratumoral expression level of VEGF or VEGFR1 gene in a cell or tissue sample of the corresponding cancer isolated from the patient, wherein the presence of:

(a) a high or overexpression of VEGF or VEGF gene expression level higher than a predetermined first value; or

(b) a high or overexpression of VEGFR1 or VEGFR1 gene expression level higher than a predetermined second value,

identifies the patient as more likely to experience tumor recurrence, or the presence of neither of (a) or (b) identifies the patient as less likely to experience tumor recurrence. In some embodiments, the presence of:

(c) a low or underexpression of VEGF or VEGF gene expression level lower than the predetermined first value; or

(d) a low or underexpression of VEGFR1 gene expression level lower than the predetermined second value,

identifies the patient as less likely to experience tumor recurrence. In some embodiments, a patient as more likely to experience tumor recurrent is as compared to a patient having a same cancer and having a low or underexpression of VEGF or VEGF gene expression level lower than the predetermined first value or a low or underexpression of VEGFR1 gene expression level lower than the predetermined second value. In some embodiments, a patient as less likely to experience tumor recurrent is as compared to a patient having a same cancer and having a high or overexpression of VEGF or VEGF gene expression level higher than a predetermined first value or a high or overexpression of VEGFR1 or VEGFR1 gene expression level higher than a predetermined second value.

In one aspect, the method identifies a patient as more likely to experience tumor recurrence the VEGF gene expression level is high or overexpressed or higher than the predetermined first value or alternatively, when a VEGFR1 gene expression level higher than the predetermined second value.

In one particular aspect, the method is used to determine if the patient is more likely to experience a shorter time to tumor recurrence than patients having the adjuvant cancer and having a VEGF gene expression level that is low or underexpressed or lower than the predetermined first value, or a VEGFR1 gene expression level that is low or underexpressed or lower than the predetermined second value.

In a further aspect, the method is used to determine if the patient as less likely to experience tumor recurrence when a VEGF gene expression level that is low or underexpressed or is lower than the predetermined first value, or a VEGFR1 gene expression level that is low or underexpressed or lower than the predetermined second value.

Also provided by this invention is a method for identifying an adjuvant cancer patient as more or less likely to experience tumor recurrence, comprising, or alternatively consisting essentially of, or yet further consisting of, determining an intratumoral expression level of VEGF gene in a cell or tissue sample of the corresponding cancer isolated from the patient, wherein a VEGF gene expression level that is high or overexpressed or higher than a predetermined value identifies the patient as more likely to experience tumor recurrence, or a VEGF gene expression level that is low or underexpressed or lower than the predetermined value identifies the patient as less likely to experience tumor recurrence.

In one aspect, the method is used to identify a patient likely to experience a shorter time to tumor recurrence than patients having the adjuvant cancer and having a VEGF gene expression level that is low or underexpressed or lower than the predetermined value.

Yet further provided is a method for identifying an adjuvant cancer patient as more or less likely to experience tumor recurrence, comprising, or alternatively consisting essentially of, or yet further consisting of, determining an intratumoral expression level of VEGFR1 gene in a cell or tissue sample of the corresponding cancer isolated from the patient, wherein a VEGFR1 gene expression level that is high or overexpressed or higher than a predetermined value identifies the patient as more likely to experience tumor recurrence, or a VEGFR1 gene expression level that is low or underexpressed or lower than the predetermined value identifies the patient as less likely to experience tumor recurrence.

In one aspect, the patient is more likely to experience tumor recurrence or likely to experience a shorter time to tumor recurrence than patients having the adjuvant cancer and having a VEGFR1 gene expression level that is low or underexpressed or lower than the predetermined value.

Briefly and for the purpose of illustration only, one of skill in the art can determine the first and second predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

The methods of this invention are useful for the prognosis and treatment of patients suffering from at least one or more cancer of the group: metastatic and non-metastatic rectal cancer, metastatic and non-metastatic colon cancer, metastatic and non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection.

Suitable samples for use in the methods of this invention include, but are not limited to a fixed tissue, a frozen tissue, a biopsy tissue, a resection tissue, a microdissected tissue, or combinations thereof.

Methods to determine gene expression level are known in the art and briefly described herein. Non-limiting examples of these methods include a method that comprises, or alternatively consists essentially of, or yet further consists of, determining the amount of mRNA transcribed from the gene, in situ hybridization, PCR, real-time PCR, or microarray. The methods are useful in the assistance of a patient such as an animal, a mammal or yet further a human patient. For the purpose of illustration only, a mammal includes but is not limited to a simian, a murine, an ovine, an equine, a canine, a bovine, a porcine or a human patient. In one aspect, the adjuvant patients are stage 2 cancer patients and had not yet received any additional therapy after surgery or surgical resection. In an alternative aspect, the adjuvant patients are stage 3 cancer patients and will receive or had received additional therapy after surgery or surgical resection.

As alternate embodiments of each of the above noted inventions, the suitable patient sample comprises, or alternatively consists essentially of, or yet further consists of, tissue or cells selected from non-metastatic tumor tissue, a non-metastatic tumor cell, metastatic tumor tissue or a metastatic tumor cell. In another aspect the patient sample can be normal tissue isolated adjacent to the tumor.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. Nos.: 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. Nos. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. Nos. 5,576,180; 6,106,777 or 7,179,600) A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. Nos. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. Nos.: 6,811,982; 6,835,541 or 7,125,673). DNA is first treated with sodium bisulfite, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. Nos.: 5,882,856; 6,531,282 or 7,118,867). By targeting multiple genes at once, additional information may be gained from a single test run that otherwise would require several times the reagents and more time to perform. Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. Nos.: 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. Nos.: 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. Nos.: 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₘ of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule-quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genes of interest identified herein. The prognostic panel comprises probes or primers that can be used to amplify and/or for determining the molecular structure of the VEGF and/or VEGFR1 gene identified herein. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the TS gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Kits

As set forth herein, the invention provides diagnostic methods for determining the expression level of VEGF and/or VEGFR1. In some embodiments, the methods use probes or primers comprising nucleotide sequences which are complementary to the gene of interest. Accordingly, the invention provides kits for performing these methods as well as instructions for carrying out the methods of this invention. Thus, in one aspect, this invention also provides a kit for use in identifying an adjuvant cancer patient more likely to have tumor recurrence, comprising, or alternatively consisting essentiallyof, or yet further consisting of, suitable primers or probes or a microarray for determining an expression level of VEGF and/or VEGFR1 gene, and instructions for use therein. Examples of suitable primers and probes are provided herein.

In one aspect, the components and instructions of the kit identifies a patient as more likely to experience tumor recurrence the VEGF gene expression level that is high or overexpressed or is higher than the predetermined first value or alternatively, when a VEGFR1 gene expression level that is high or overexpressed or higher than the predetermined second value.

In one particular aspect, the components and instructions of the kit is used to determine if the patient is more likely to experience a shorter time to tumor recurrence than patients having the adjuvant cancer and having a VEGF gene expression level that is low or underexpressed or lower than the predetermined first value, or a VEGFR1 gene expression level that is low or underexpressed or lower than the predetermined second value.

In a further aspect, the components and instructions of the kit is used to determine if the patient as less likely to experience tumor recurrence when a VEGF gene expression level that is low or underexpressed or is lower than the predetermined first value, or a VEGFR1 gene expression level that is low or underexpressed or lower than the predetermined second value.

Also provided by this invention are the components and instructions of the kit for identifying an adjuvant cancer patient more likely to experience tumor recurrence, comprising, or alternatively consisting essentially of, or yet further consisting of, determining an intratumoral expression level of VEGF gene in a cell or tissue sample of the corresponding cancer isolated from the patient, wherein a VEGF gene expression level that is high or overexpressed or higher than a predetermined value identifies the patient as more likely to experience tumor recurrence, or a VEGF gene expression level that is low or underexpressed or lower than the predetermined value identifies the patient as less likely to experience tumor recurrence.

In one aspect, the method is used to identify a patient likely to experience a shorter time to tumor recurrence than patients having the adjuvant cancer and having a VEGF gene expression level that is low or underexpressed or lower than the predetermined value.

Yet further provided are the components and instructions of the kit for identifying an adjuvant cancer patient more likely to experience tumor recurrence, comprising, or alternatively consisting essentially of, or yet further consisting of, determining an intratumoral expression level of VEGFR1 gene in a cell or tissue sample of the corresponding cancer isolated from the patient, wherein a VEGFR1 gene expression level that is high or overexpressed or higher than a predetermined value identifies the patient as more likely to experience tumor recurrence, or a VEGFR1 gene expression level that is low or underexpressed or lower than the predetermined value identifies the patient as less likely to experience tumor recurrence.

In one aspect, the patient is more likely to experience tumor recurrence or likely to experience a shorter time to tumor recurrence than patients having the adjuvant cancer and having a VEGFR1 gene expression level lower than the predetermined value.

Briefly and for the purpose of illustration only, one of skill in the art can determine the first and second predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

The components and instructions of the kit are useful for the prognosis and treatment of patients suffering from at least one or more cancer of the group: metastatic and non-metastatic rectal cancer, metastatic and non-metastatic colon cancer, metastatic and non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection. In one aspect, the adjuvant patients are stage 2 cancer patients and had not yet received any additional therapy after surgery or surgical resection. In an alternative aspect, the adjuvant patients are stage 3 cancer patients and will receive or had received additional therapy after surgery or surgical resection. The additional therapy comprises, or alternatively consists essentially of, or yet further consists of 5-FU based adjuvant therapy.

The methods are useful in the assistance of an animal, a mammal or yet further a human patient. For the purpose of illustration only, a mammal includes but is not limited to a simian, a murine, a bovine, an equine, a porcine or an ovine.

Suitable samples for use in the methods of this invention include, but are not limited to a fixed tissue, a frozen tissue, a biopsy tissue, a resection tissue, a microdissected tissue, or combinations thereof.

Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, blood, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S. and Thompson, eds., (1991) GENETICS IN MEDICINE, W B Saunders Co., Philadelphia, Pa. This is useful, e.g., in forensic studies.

### Therapeutic Methods

Also provided by this invention is a method for treating a patient having a cancer by administering to the patient a therapy comprising, or alternatively consisting essentially of, or yet further consisting of an adjuvant cancer therapy, wherein the patient is selected for the therapy based on a genotype of low or underexpression of VEGF or VEGF gene expression level lower than the predetermined value identifies in a sample isolated from the patient, thereby treating the patient.

Also provided is a method for treating a patient having cancer by administering by to the patient a therapy comprising, or alternatively consisting essentially of, or yet further consisting of an adjuvant cancer therapy, wherein the patient is selected for the therapy based on a genotype of low or underexpression of VEGFR1 or VEGFR1 gene expression level lower than the predetermined value identifies the patient in a sample isolated from the patient, thereby treating the patient.

The methods of this invention are useful for the treatment of patients suffering from at least one or more cancer of the group: metastatic and non-metastatic rectal cancer, metastatic and non-metastatic colon cancer, metastatic and non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection.

Suitable samples for use in the methods of this invention include, but are not limited to a fixed tissue, a frozen tissue, a biopsy tissue, a resection tissue, a microdissected tissue, or combinations thereof.

Methods to determine gene expression level are known in the art and briefly described herein. Non-limiting examples of these methods include a method that comprises, or alternatively consists essentially of, or yet further consists of, determining the amount of mRNA transcribed from the gene, in situ hybridization, PCR, real-time PCR, or microarray. The methods are useful in the assistance of a patient such as an animal, a mammal or yet further a human patient. For the purpose of illustration only, a mammal includes but is not limited to a simian, a murine, an ovine, an equine, a canine, a bovine, a porcine or a human patient. In one aspect, the adjuvant patients are stage 2 cancer patients and had not yet received any additional therapy after surgery or surgical resection. In an alternative aspect, the adjuvant patients are stage 3 cancer patients and will receive or had received additional therapy after surgery or surgical resection.

As alternate embodiments of each of the above noted inventions, the suitable patient sample comprises, or alternatively consists essentially of, or yet further consists of, tissue or cells selected from non-metastatic tumor tissue, a non-metastatic tumor cell, metastatic tumor tissue or a metastatic tumor cell. In another aspect the patient sample can be normal tissue isolated adjacent to the tumor.

The invention now being generally described, it will be more readily understood by reference to the following example which is included merely for purposes of illustration of certain aspects and embodiments of the present invention, and are not intended to limit the invention.

### Experimental Details

### EXAMPLE 1

Background: Tumor recurrence after curative resection is still a major problem in the management of adjuvant colon cancer, with recurrence rate approximately 30-40%. Identifying molecular markers for tumor recurrence is critical for successfully selecting patients who are more likely to benefit from adjuvant chemotherapy. Here it was tested whether gene expression levels of angiogenesis pathway (COX-2, EGFR, VEGF, VEGFR1, VEGFR2 and IL-8) could predict the risk of tumor recurrence in stage II and III colon cancer patients treated with adjuvant chemotherapy.

Methods: Tissue samples from 140 adjuvant colon cancer patients (69 females and 71 males with a median age of 59 years; range=28-86) were available for gene expression assays. These tissue samples were obtained at the University of Southern California/Norris Comprehensive Cancer Center (USC/NCCC) and LAC+USC medical center between 1999 and 2006. Sixty-three patients had stage II and 77 had stage III colon cancer. The median follow-up was 5.4 years (range=2.0-16.8). 51 of 140 patients (36.4%) developed tumor recurrence with a 5-year probability of 0.28±0.06 for stage II and 0.40±0.06 for stage III colon cancer patients. mRNA was extracted from laser-capture-microdissected tumor tissue. After cDNA was prepared by reverse transcription, quantitation of the candidate genes and an internal reference gene (B-actin) was performed using a fluorescence-based real-time detection method (TaqMan®). Primers used in the real-time detection method are included in Table 1.

TABLE 1
Primers used in real-time PCR

| | Forward Primer | Reverse Primer | Taqman Probe |
|---|---|---|---|
| Gene | (5′-3′) | (5′-3′) | (5′-3′) |
| β-actin | GAGCGCGGCTACAGCTT | TCCTTAATGTCACGCAC | ACCACCACGGCCGAGCGG |
| | (SEQ ID NO. 1) | GATTT | (SEQ ID NO. 3) |
| | | (SEQ ID NO. 2) | |
| VEGF | AGTGGTCCCAGGCTGCAC | TCCATGAACTTCACCAC | ATGGCAGAAGGAGGAGGG |
| | (SEQ ID NO. 4) | TTCGT | CAGAATCA |
| | | (SEQ ID NO. 5) | (SEQ ID NO. 6) |
| VEGF | CGCATATGGTATCCCTCA | AGTCACACCTTGCTTCG | TGGTTCTGGCACCCCTGTA |
| R1 | ACCT | GAATG | ACCATAA |
| | (SEQ ID NO. 7) | (SEQ ID NO. 8) | (SEQ ID NO. 9) |

Results: It was found that VEGF and VEGFR1 gene expression levels independently significantly associated with time to tumor recurrence in adjuvant colon cancer patients. Patients with lower VEGF gene expression and lower VEGFR1 gene expression levels had significantly longer time to tumor recurrence compared to those with higher VEGF and higher VEGFR1 gene expression levels (p<0.05, log-rank test).

Conclusions: VEGF and VEGFR1 gene expression levels can predict tumor recurrence risk in adjuvant colon cancer patients.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying an adjuvant cancer patient as more or less likely to experience tumor recurrence, comprising determining an intratumoral expression level of VEGF or VEGFRI gene in a cell or tissue sample of the corresponding cancer isolated from the patient, wherein the presence of:
(a) a VEGF gene expression level higher than a predetermined first value; or
(b) a VEGFRI gene expression level higher than a predetermined second value, identifies the patient as more likely to experience tumor recurrence, or the presence of neither of (a) or (b) identifies the patient as less likely to experience tumor recurrence.

2. The method of claim 1, wherein the presence of:
(a) a VEGF gene expression level higher than a predetermined first value; or
(b) a VEGFRI gene expression level higher than a predetermined second value, identifies the patient as more likely to experience tumor recurrence.

3. The method of claim 1, wherein a patient more likely to experience tumor recurrence is a patient having a shorter time to tumor recurrence than a patient having the adjuvant cancer and having neither of (a) or (b).

4. The method of claim 1, wherein the presence of neither of (a) or (b) identifies the patient as less likely to experience tumor recurrence.

5. The method of claim 1, wherein a patient less likely to experience tumor recurrence is a patient having a longer time to tumor recurrence than a patient having the adjuvant cancer and having a VEGF gene expression level lower than the predetermined first value, or a VEGFR1 gene expression level lower than the predetermined second value.

6. A method for identifying an adjuvant cancer patient as more or less likely to experience tumor recurrence, comprising determining an intratumoral expression level of VEGF gene in a cell or tissue sample of the corresponding cancer isolated from the patient, wherein a VEGF gene expression level higher than a predetermined value identifies the patient as more likely to experience tumor recurrence, or a VEGF gene expression level lower than the predetermined value identifies the patient as less likely to experience tumor recurrence.

7. The method of claim 6, wherein a patient more likely to experience tumor recurrence is a patient having a shorter time to tumor recurrence than a patient having the adjuvant cancer and having a VEGF gene expression level lower than the predetermined value.

8. A method for identifying an adjuvant cancer patient as more or less likely to experience tumor recurrence, comprising determining an intratumoral expression level of VEGFR1 gene in a cell or tissue sample of the corresponding cancer isolated from the patient, wherein a VEGFR1 gene expression level higher than a predetermined value identifies the patient as more likely to experience tumor recurrence, or a VEGFR1 gene expression level lower than the predetermined value identifies the patient as less likely to experience tumor recurrence.

9. The method of claim 8, wherein a patient is more likely to experience tumor recurrence is a patient having a shorter time to tumor recurrence than a patient having the adjuvant cancer and having a VEGFR1 gene expression level lower than the predetermined value.

10. The method of claim 1, wherein the colon cancer sample is at least one of a fixed tissue, a frozen tissue, a biopsy tissue, a resection tissue, a microdissected tissue, or combinations thereof.

11. The method of claim 1, wherein the gene expression level is determined by a method that comprises determining the amount of mRNA transcribed from the gene.

12. The method of claim 1, wherein the gene expression level is determined by a method comprises one or more of in situ hybridization, PCR, real-time PCR, or microarray.

13. The method of claim 1, wherein the adjuvant cancer patient suffered from at least one cancer of the type of the group head and neck cancer, metastatic and non-metastatic rectal cancer, metastatic and non-metastatic colon cancer, metatstatic and non-metastatic colorectal cancer, lung cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection.

14. The method of claim 1, wherein the adjuvant cancer patient suffered from colon cancer prior to a surgical resection.

15. The method of claim 1, wherein the patient is a human patient.

16.-24. (canceled)

25. A panel of probes and/or primers and/or a microarray to determine an intratumoral expression level of VEGF and VEGFR1 genes in a cell or tissue sample.


SEQUENCE LISTING (9 sequences)

SEQ ID NO: 1 - DNA, 17 bp, Artificial Sequence
  Description of Artificial Sequence Synthetic primer
gagcgcggctacagctt

SEQ ID NO: 2 - DNA, 22 bp, Artificial Sequence
  Description of Artificial Sequence Synthetic primer
tccttaatgtcacgcacgattt

SEQ ID NO: 3 - DNA, 18 bp, Artificial Sequence
  Description of Artificial Sequence Synthetic probe
accaccacggccgagcgg

SEQ ID NO: 4 - DNA, 18 bp, Artificial Sequence
  Description of Artificial Sequence Synthetic primer
agtggtcccaggctgcac

SEQ ID NO: 5 - DNA, 22 bp, Artificial Sequence
  Description of Artificial Sequence Synthetic primer
tccatgaacttcaccacttcgt

SEQ ID NO: 6 - DNA, 26 bp, Artificial Sequence
  Description of Artificial Sequence Synthetic probe
atggcagaaggaggagggcagaatca

SEQ ID NO: 7 - DNA, 22 bp, Artificial Sequence
  Description of Artificial Sequence Synthetic primer
cgcatatggtatccctcaacct

SEQ ID NO: 8 - DNA, 22 bp, Artificial Sequence
  Description of Artificial Sequence Synthetic primer
agtcacaccttgcttcggaatg

SEQ ID NO: 9 - DNA, 26 bp, Artificial Sequence
  Description of Artificial Sequence Synthetic probe
tggttctggcacccctgtaaccataa